(19) United States
(12) Patent Application Publication
(10) Pub. No.: US 2008/0118551 A1
(43) Pub. Date: May 22, 2008

(54) Denatured human albumin biomaterial as dressing for burns and wounds of skin

(76) Inventor: Yasmin Wadia, Humble, TX (US)

Correspondence Address: ELIZABETH R. HALL, 1722 MARYLAND STREET, HOUSTON, TX, 77006, US

(21) Appl. No.: 11/985,475

(22) Filed: Nov. 14, 2007

Related U.S. Application Data
(60) Provisional application No. 60/866,571, filed on Nov. 20, 2006.

(51) Int. Cl. A61K 35/12; A61K 9/70

(52) U.S. Cl. 424/445; 424/93.7

(57) ABSTRACT

The present invention provides biocompatible burn and wound dressing materials that are made from denatured human serum albumin. The burn and wound dressing materials can be constructed by molding of liquid human serum albumin into sheets or desired shapes. The burn and wound dressing materials can be impregnated with a variety of agents that are used to promoted burn and wound healing. The denatured albumin wound dressing material is biocompatible and biodegradable and once applied to the wound does not require being removed from the wound. The wound dressing material is penetrated with visible, ultraviolet and infrared radiation.

### CROSS-REFERENCE TO RELATED APPLICATION

The present application, pursuant to 35 U.S.C. 111(b), claims the benefit of the earlier filing date of provisional application Ser. No. 60/866,571 filed Nov. 20, 2006, and entitled “Denatured Human Albumin Biomaterial as Burns, Wound Dressing and Sutures, Applications and Manufacture.”

### BACKGROUND OF THE INVENTION

1. Field of the Invention

The invention relates generally to biocompatible skin wound and burn dressing materials and uses of the material to repair skin defects and to promote wound healing. More particularly, the present invention relates to a skin wound and burn dressing material containing denatured human serum albumin.

2. Description of the Related Art

The skin serves as a protective barrier against the environment. The skin serves as a barrier to infection and prevents the loss of water and electrolytes from the body. Thus, the loss of the integrity of large portions of the skin as a result of illness or injury can lead to major disability or even death.

Every year in the United States there are 1.1 million burn patients who require medical attention and 6.5 million patients are reported to have chronic skin ulcers caused by pressure, venous stasis, or diabetes mellitus. Thus, acceleration of skin wound healing has been an active area of medical research and improved designs of skin repair materials have been sought for decades.

Skin wound healing is a dynamic, interactive process involving soluble mediators, blood cells, extracellular matrix, and parenchymal cells. From the metabolic perspective, skin wound healing requires deposition of new proteins to repair tissue defects and formation of new epidermis to cover the surface. These metabolic events in the skin wound are regulated by nutrients, hormones and growth factors (Kloth, L. C. and J. M. McCulloch (eds). 2002. Wound Healing: Alternatives in Management. Philadelphia: FA Davis Co., pp. 1-147; Johnson, C. 1994. Burn Care and Rehabilitation: Principle and Practice. Philadelphia: FA Davis Co., pp. 29-33; Clark, R. A. F. 1998. Burn Care and Rehabilitation: Principle and Practice. Philadelphia: FA Davis Co., pp. 3-13).

Wound metabolism has been defined as phases (ebb, turn and flow) of responses that occur following trauma and is seen at both the systemic and local levels (Cuthberson, D. P. et al. 1972. Nutr. Metab. 14:92-109). The “ebb” phase refers to depressed vitality of biological activity, reflected by low heat production and substrate kinetics with low levels of hormones and growth factors. In contrast, the “flow” phase is characterized as the hypermetabolic response with increased heat production, protein catabolism, lipolysis, and insulin resistance. The “turn” phase is that transition time between the ebb and flow phases.

The metabolic changes following major burns at the whole body level have been extensively studied. The knowledge of the metabolic responses serves as a guide to nutritional support (Hildreth, M. and M. Gottschlich. 1996. Total Burn Care. Philadelphia: WB Saunders & Co., pp. 237-246). Nonetheless, little quantitative information is available with respect to the metabolic events in local wounds.

It has been reported that protein kinetics in scalded skin were significantly increased on day seven after injury but not 48 hours after injury (Zhang, X-J. et al. 1999. Am. J. Physiol. Endocrinol. Metab. 276:E712-E720; Zhang, X.-J. et al. 2000. Am. J. Physiol. Endocrinol. Metab. 278:E452-E461). Recently it was found that protein turnover in the skin donor wound increased several-fold on day seven but not on day one or day three after injury (Zhang, X.-J. et al. 2004. J. Burn Care Rehab. 25:S148). These results confirm that day seven constitutes the flow phase, while days one through three are in the ebb phase.

The turn phase is considered as a time period between days four and six. Even though the turn phase is difficult to define, understanding the factors that initiate the turn phase may be important because a prolonged ebb phase often leads to deterioration of the patient's condition (Cuthberson, D. P. et al. 1972. Nutr. Metab. 14:92-109; Hildreth, M. and M. Gottschlich. 1996. Total Burn Care. Philadelphia: WB Saunders & Co., pp. 237-246). The turn phase is a desirable time period to investigate nutritional and hormonal effects on wound metabolism because at that time the metabolic activity in the wound has the potential to increase (Zhang, X.-J. et al. 2004. J. Burn Care Rehab. 25:S148).

In contrast with marked change in protein turnover, the rate of wound DNA synthesis has been shown to be relatively constant and close to the normal skin rate (Zhang, X.-J. et al. 2004. J. Burn Care Rehab. 25:S148; Zhang, X.-J. et al. 2004. J. Nutr. 134:2401-2406). This finding was somewhat surprising since the wound cells (keratinocytes and fibroblasts) are thought to proliferate rapidly (Kloth, L. C. and J. M. McCulloch (eds). 2002. Wound Healing Alternatives in Management. Philadelphia: FA Davis Co., pp. 1-147). Recent progress in wound healing research has demonstrated the possibility of accelerating wound healing with anabolic hormones (Demling, R. H. 2005. J. Burns Wounds 3:11).

There has been a wide variety of research into methods to enhance and control healing of wounds, including burns. Healing has been shown to be affected by altering the wound environment (oxygen levels, application of magnetic fields) or contacting wounds with compounds with known biochemical activity (growth factors, growth hormone). For skin wounds the goal of treatment is to regenerate the connective tissue and epidermis and produce healthy skin.

Biodegradable matrices have been used as one method to promote healing of skin wounds and burns. An example of such a matrix is a fibrin matrix. Fibrin has been used as a tissue adhesive for several decades. Additionally, a biodegradable scaffold matrix composed of albumin, a polyethylene glycol cross-linking agent, and polymeric beads has been developed (U.S. Pat. No. 6,656,496). This scaffold matrix has also incorporated different factors to promote tissue healing such as fibroblast growth factor I, anti-inflammatory agents, and antibiotics.

Collagen has been used as a wound dressing for tissues, including skin (U.S. Pat. Nos. 3,157,524 and 4,320,201). Yet another example of a wound dressing that has been employed is a hydrocolloid dressing (U.S. Pat. No. 3,969,498). More recently, a wound dressing device has been described that comprises a matrix of a polymer network and a non-gellable polysaccharide, where agents to promote healing can also be incorporated into the matrix (U.S. Pat. No. 6,355,858).

Artificial skin has also been developed (U.S. Pat. No. 7,244,552). None of the products developed to date is ideally suited for all types of skin burns or wounds. Each of these wound dressing materials and devices has advantages and disadvantages inherent in their chemical compositions and physical properties. There remains a continuing need for biocompatible materials that can be used to treat skin burns and wounds.

### SUMMARY OF THE INVENTION

The present invention is a biocompatible wound and burn dressing material formed from a denatured human serum albumin. In one embodiment, wound and burn dressing material is made by denaturing a solution of human serum albumin. The solution contains a concentration of from 47% to 58% human serum albumin.

In another embodiment, the biocompatible dressing material is formed by rolling liquid human serum albumin into sheets and denaturing the liquid albumin by application of wet or dry heat at a temperature of from about 85° C. to about 120° C. for from 15 seconds to about 30 minutes.

In yet another embodiment, the biocompatible dressing material is formed by molding liquid human serum albumin into a desired shape that conforms the to tissue architecture at the site of desired use. Also provided are methods for wound and burn treatment by dressing the wound or burn with the biocompatible dressing material of the present invention.

Another embodiment of the present invention is a burn and wound dressing comprising a denatured human albumin derived from denaturing a human serum albumin solution having an albumin concentration of from about 47% w/v to about 58% w/v.

Another embodiment of the present invention is a biocompatible burn and wound dressing comprising: a first sheet of dressing material including at least 47% w/v denatured human serum albumin; and a second sheet of dressing material including a topical agent.

Another embodiment of the present invention is a biocompatible burn and wound dressing comprising at least one sheet of dressing material containing a denatured human serum albumin at a concentration of at least 50% w/v, wherein the dressing material has a yield strength ranging from about 800 kilopascals to about 1200 kilopascals and a Young's modulus of elasticity ranging from about 2500 kilopascals to about 3500 kilopascals.

Another embodiment of the present invention is a biocompatible burn and wound dressing comprising: a first sheet of porous dressing material including at least 47% w/v denatured human serum albumin; and a second sheet of dressing material penetrable by visible light, ultraviolet radiation and infrared radiation.

Another embodiment of the present invention is a method for making a burn and wound dressing comprising the steps of: obtaining an albumin solution having a concentration of human serum albumin ranging from about 47% w/v to about 58% w/v; casting the albumin solution into a predetermined shape; and denaturing the human serum albumin by heating the albumin to at least 85° C. at a pressure of at least 1 atmosphere for at least 15 seconds.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts results of experiments to determine the yield strengths of albumin strips cured at various times at 100° C.

FIG. 2 depicts yield strengths for albumin sheets that have been cured at 86° C. The thickness of the sheets is given on the vertical axis in micrometers (μm); for example, 270b means that the sample was the second sample with a thickness of 270 μm.

FIG. 3 depicts yield strengths of albumin strips cured at multiple temperatures. Also included are results with autoclaved material.

FIG. 4 depicts the Young's modulus (kPa) for albumin sheets that have been cured at 86° C. The thickness of the sheets is given on the vertical axis in μm; for example 270b means that the sample was the second sample with a thickness of 270 μm.

FIG. 5 depicts the Young's modulus (kPa) for albumin strips cured at 100° C. showing the stiffness of the albumin strips increases with increased curing times

FIG. 6 depicts embodiments of the present invention having a smooth, textured, or fenestrated surface texture.

FIG. 7 depicts one example of a sandwiched dressing material that can be manufactured using the denatured albumin materials of the present invention.

### DESCRIPTION OF THE PREFERRED EMBODIMENTS

The present invention relates to a biocompatible, bioabsorbable burn and wound dressing material containing denatured human serum albumin. The dressing material is formed by denaturing a solution of human serum albumin.

Denatured Human Serum Albumin

Denatured human serum albumin has been recognized as a safe and effective biomaterial with a variety of applications. It has been employed as a food additive and fat replacement agent (U.S. Pat. No. 7,166,316), as a material for construction of drug delivery devices (U.S. Pat. No. 4,666,641), and as a component of implantable materials in order to inhibit thrombogenesis (U.S. Pat. No. 5,632,776). These applications of denatured human serum albumin demonstrate the versatility of the substance as well as the biocompatibility of the basic product. The U.S. Food and Drug Administration has approved human serum albumin as safe and effective in some of these applications.

U.S. Pat. No. 6,680,063 B1 discloses a denatured human serum albumin composition and methods for making the composition, as well as application of the denatured albumin compositions for repair of tissue defects and lesions. The denatured human serum albumin product of this patent is formed into sheets that can be then molded or cut by a surgeon as needed during surgery for tissue repair. The denatured albumin compositions contained from 50% to 58% albumin and comprises a thin, pliable sheet formed to a thickness of from 75 μm to about 300 μm. The patent also discloses applying the biocompatible denatured albumin material to a solid visceral organ along with an energy-absorbing proteinaceous material and irradiating the materials in order to fuse the denatured albumin and the proteinaceous material to the tissue for tissue repair. The patent discloses that the denatured human albumin sheets have the strength for tissue repair and the biocompatibility required for internal organ exposure, while allowing for efficient wound healing.

Human serum albumin is a composition that has been approved for human use by the U.S. Food and Drug Administration (FDA). FDA-approved human serum albumin has been established to be completely biocompatible and biodegradable. Unlike other biomaterials that are often derived from animal sources (e.g., collagen, elastin), human-derived biomaterials such as human serum albumin have less concern in terms of potential antigenicity, immune rejection and foreign body reactions. The term “biocompatible material” is used herein to mean that a material does not initiate an antigenic reaction or an immune rejection when used to treat wounds or burns in humans.

Furthermore, the use of human source materials avoid concerns attendant with animals such as transmission of animal diseases and viruses. Since the protein sequence and structure of human serum albumin varies little among the human population, human serum albumin is a preferred biomaterial.

The denatured albumin burn and wound dressing of the present invention has many desirable characteristics that enhance the utility of the dressing. For example, the denatured albumin material can be prepared and then stored at room temperature. Once applied, the burn and wound dressing can be left in place for up to 14 days. The denatured albumin material is clear and transmits visible light and ultraviolet light, thereby allowing for the visual inspection of the surface wound without removing the dressing, as well as allowing for UV light therapy to reach the burn area without removal of the dressing.

The denatured albumin material is non-antigenic and prevents water loss at the site of application, as well as providing a barrier to bacteria. Moreover the denatured albumin can be impregnated with desired antibiotics or growth factors as needed in order to promote healing of the skin.

The pliability of the denatured albumin material means that it drapes well over a wound and is easy to secure to the skin surface. The denatured albumin material can be applied in one operation, does not allow hypertrophic granulation tissue to form, and helps prevent contracture of the wound surface.

Experiments have been performed to demonstrate some of these characteristics of the denatured albumin material. The results of such experiments are described below.

It is contemplated that the denatured albumin burn and wound dressing of the present invention will benefit patients suffering from vesicant burns and thermal burns, including first degree burns, second degree burns and third degree burns, as well as esophageal burns and erosions. It is also contemplated that the denatured albumin burn and wound dressing of the present invention will benefit patients with chronic skin ulcers, including but limited to decubitus ulcers, venous stasis ulcers, arterial insufficiency ulcers, and diabetic foot ulcers. The injuries that are contemplated to be treated by use of the burn and wound dressing of the present invention include any denuded area without skin or mucosa that is due to trauma such as a chemical burn, a radiation burn, a thermal burn, an excision trauma, a surgical trauma, an abrasion, or due to a malignancy, an infection, or an allergic reaction (i.e., Steven Johnson Syndrome). It is believed that the use of the denatured albumin material of the present invention will result in an improved cosmetic and functional outcome for patients.

It has previously been shown that denatured albumin can be formed into a sheet or lamina structure (U.S. Pat. No. 6,680,063 B1). The denatured lamina is clear, flexible, thin, and can be prepared at a uniform thickness. Once prepared in sheets, the composition can be easily manipulated without special care due to its high tensile strength and pliability. Although the surface of the sheet has a slight tackiness, the material does not bond or stick to itself in a manner to interfere with its use in vivo.

Denaturation of the albumin results in a lamina that is stable in a variety of environments, including those that are characteristic of human physiology. The denatured lamina does not solubilize in water or saline solution, nor after contact with tissue. These properties allow the lamina to be repositioned after initial contact with tissue.

The denatured albumin lamina is stable for certain periods of time in air, remaining pliable for as long as 15 minutes when exposed to air. As a result, the denatured albumin product is stored under vacuum once manufactured, until use.

The denatured lamina is typically sterilized using an autoclave, gamma irradiation, or ethylene oxide gas. Because denaturation is desired, autoclaving the material can concurrently accomplish both the sterilization and denaturation steps in the manufacture of the lamina.

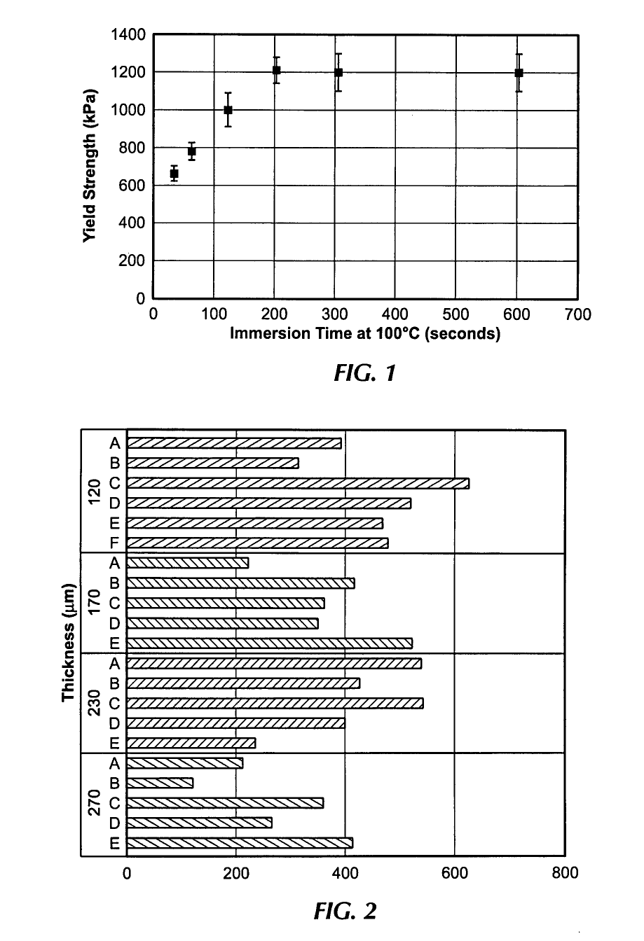

Studies have been performed to test the strength of the denatured albumin lamina. Albumin lamina was cured at 100° C. for 30, 60, 120, 200, 300, or 600 seconds in which a dog bone pattern die was used to cut albumin strips so that the failure point was consistently in the middle of the sample, rather than at the clamps. Strips of approximately 2×1 centimeter (cm) were stretched in a Chatillon Materials Tester. Ultimate strength, or yield strength, was calculated for the albumin strips by dividing the cross-sectional area of the test strip into the force required to break the test strip (FIG. 1). The results showed that yield strengths increased almost linearly with cure times from 30-200 seconds. Curing the albumin strips for longer than 200 seconds, however, did not significantly increase the yield strength of the material.

Ultimate strengths were also determined for albumin strips of different thicknesses cured at 86° C. for 30 seconds (FIG. 2). The albumin strips were cut from the albumin sheets with a dog bone pattern die so that the failure point was consistently in the middle of the sample, rather than at the clamps. The ultimate strengths were recorded along with the exact width and thickness of each sample. The ultimate strength was calculated by dividing the force required to break the sample by the cross-sectional area (width×thickness).

Multiple strips having a thickness of 120 μm, 170 μm, 230 μm, and 270 μm were tested. FIG. 2 shows the thickness of each strip on the vertical axis and the yield strength of each strip along the horizontal axis. The results for six denatured albumin strips having a thickness of 120 μm (labeled A-F), five albumin strips having a thickness of 170 μm (labeled A-E), five denatured albumin strips having a thickness of 230 μm (labeled A-E), and five denatured albumin strips having a thickness of 270 μm (labeled A-E) are shown in FIG. 2.

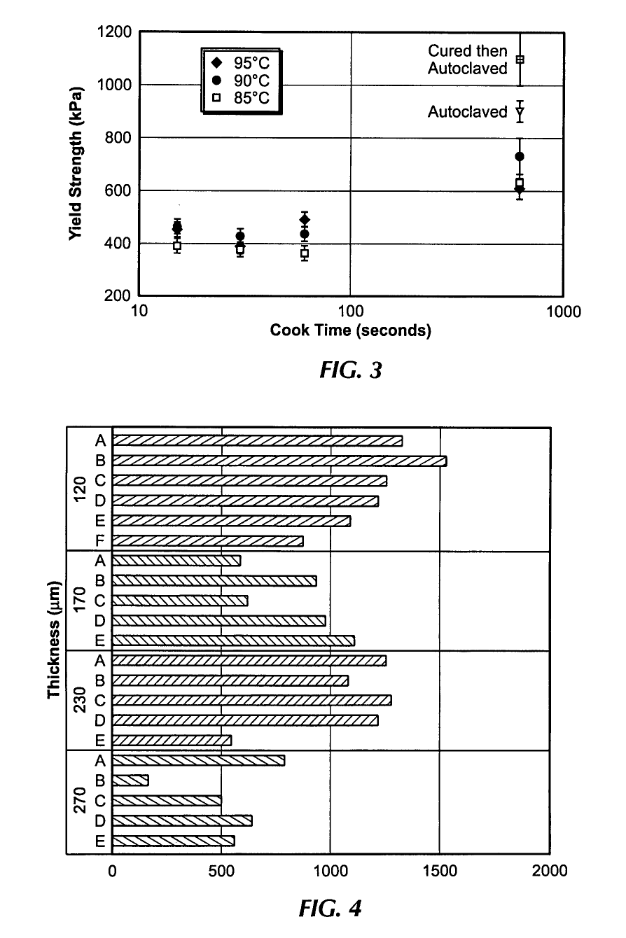

FIG. 3 illustrates the ultimate strengths in kilopascals (kPa) of albumin strips denatured by heat bath immersion at 85° C., 90° C., and 95° C. The ultimate strengths of two sets of autoclaved albumin strips were also measured for comparison. Once set of autoclaved materials was cured by autoclaving the material at 110° C. and the other set was initially cured by a 15-30 second heat bath immersion and then autoclaved at 110° C. The data illustrate a significant increase in the yield strength of the autoclaved denatured albumin strips and an insignificant increase in the yield strength of the denatured albumin strips cured at 85° C., 90° C., and 95° C. Furthermore, the results clearly illustrate that there was a significant increase in yield strength of the denatured albumin strips cured for 600 seconds versus the denatured albumin strips that were cured from 15 to 60 seconds.

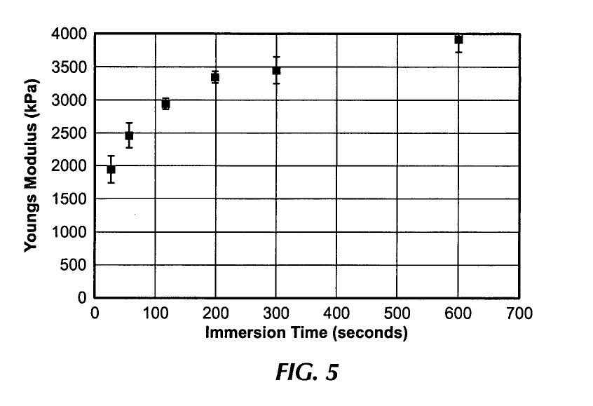

Experiments were also performed to test the elasticity of denatured albumin strips cured at 86° C. (FIG. 4) and 100° C. (FIG. 5). Young's modulus of elasticity was calculated for each sample by a linear fit of stress/strain data for strains ranging from 0 to 0.1.

The elasticity or stiffness of the denatured albumin strips of different thicknesses cured at 86° C. for 30 seconds was determined (FIG. 4). The Young's modulus of elasticity were recorded for multiple denatured albumin strips having a thickness of 120 μm, 170 μm, 230 μm, and 270 μm. FIG. 4 shows the thickness of each strip on the vertical axis and the Young's modulus (kPa) of each strip along the horizontal axis. The results for six denatured albumin strips having a thickness of 120 μm (labeled A-F), five denatured albumin strips having a thickness of 170 μm (labeled A-E), five denatured albumin strips having a thickness of 230 μm (labeled A-E), and five denatured albumin strips having a thickness of 270 μm (labeled A-E) are shown in FIG. 4.

Studies were performed to test the elasticity of denatured albumin strips cured for different time periods. Albumin strips were cured at 100° C. for 30, 60, 120, 200, 300, or 600 seconds. Young's modulus of elasticity was calculated for each sample by a linear fit of stress/strain data for strains ranging from 0 to 0.1. The results showed that the stiffness (Young's modulus) of the denatured albumin strips increased with increased curing time, with the majority of the effect seen within the first 200 seconds.

The data on strength and elasticity provided parameters for developing denatured albumin burn and wound dressing materials. Different configurations of the denatured albumin burn and wound dressing materials were designed for different applications. Some of the different configurations of the materials utilize different mechanical properties of strength and elasticity. However, typically the burn and wound dressing materials have a yield strength of at least 400 kPa (preferably ranging from about 800 kPa to about 1200 kPa) and an elasticity of less than 4000 kPa (preferably ranging from about 2500 kPa to about 3500 kPa).

Any method of manufacture that produces a thin sheet or lamina of denatured human serum albumin material is contemplated by the present invention. However, one preferred method of manufacture, described below, involves the preparation of denatured albumin sheets or lamina of varying thickness.

The starting material for the preparation of the lamina is a liquid human serum albumin solution of approximately 47% to 58% albumin concentration. As used hereinafter, the terms “percent” and “%” refer to weight per volume (gm/100 ml) unless otherwise noted.

The concentrated albumin solution is placed between two nonporous sheets (e.g., medical grade plastic or preferably PTFE or Teflon®). Typically the concentrated albumin solution is continuously placed between the two aligned sheets. The sheets are then rolled through graduated rollers to spread the albumin evenly and to a uniform thickness of from about 50 μm to about 500 μm. The rolled liquid albumin between the non porous sheets is subject to wet or dry heat ranging from about 86° C. to about 120° C. for 15-200 seconds which denatures the liquid albumin to form a solid.

Alternatively, sheets are cured at about 90° C. for about 15 seconds and then autoclaved at 110° C. for about 10 minutes. The liquid albumin may also be denatured by autoclaving alone. The factors shown to influence the properties of the resultant denatured lamina are the concentration of human serum albumin in the albumin solution (47% to 58%), curing temperature (85° to 120° C.), curing time (15 seconds to 10 minutes), and curing pressure (1 atm to 3 atm). The liquid albumin is preferably denatured at 100° C. for 120 seconds at a pressure of about 2 atmospheres.

Using this basic manufacturing method for denatured albumin materials, the denatured albumin materials can be prepared in different configurations as described below.

Testing of the denatured albumin lamina has been performed to examine various properties of the material that would be critical to the application of the material to manufacture of burn and wound dressing materials. Such testing is required by regulatory agencies during development of wound dressing materials.

One such type of testing, cytotoxicity testing, is an important consideration for any material that is in contact with human tissues, which would include burn and wound and dressing materials. In the present invention, cytotoxicity testing was performed on samples of denatured albumin in accordance with Good Laboratory Practice (GLP) regulations. The test performed, a MEM elution test, is a standard test procedure performed on all types of medical devices, including wound dressing materials. Cytotoxicity testing is an in vitro test process that is a rapid and sensitive method to determine if the materials used contain significant quantities of harmful extractables, and then to quantify the effect of such extractables on cellular components.

In the MEM elution test, a test sample of extracted denatured albumin was placed in contact with a monolayer of mouse heteroploid connective tissue (L-929) cells and then incubated. The cells were then scored for cytotoxic effects (degree of cellular destruction).

More specifically, an extract of denatured albumin was prepared based on USP and ANSI/AAMI/ISO surface area recommendations or weight. The sample was extracted for 24 to 25 hours at 37° C. in 1×Minimal Essential Media with 5% calf serum. Positive (latex natural rubber) and negative (polypropylene pellets) controls were similarly extracted and included in the assay. A blank of extraction media (a “media control”) was also included in the assay. Multiple well cell culture plates were seeded with a verified quantity of L-929 cells and incubated until 80-90% confluent. The cell culture media was removed from the plates. The test extracts were filtered and the appropriate amount of extract was added to each well on the cell culture plates. Each extract was tested on three wells of cells. The cells were incubated at 37° C. with 5±1% CO² for 72±3 hours.

The cell monolayers were examined microscopically. The wells were scored as to the degree of discernable morphological cytotoxicity on a relative scale of 0 to 4 (0=no reactivity; 1=slight reactivity; 2=mild reactivity; 3=moderate reactivity; 4=severe reactivity). The results from the three wells were averaged to give a final cytotoxicity score.

The results showed that the denatured albumin extract samples exhibited mild activity only (score of grade 2 of 4) in the MEM elution test. Using the standards set forth by the United States Pharmacopeia (USP), the denatured albumin material is acceptable for human contact (i.e., a score no greater than 2).

In addition to the in vitro testing for cytotoxicity, in vivo tests of biocompatibility were also performed with the denatured albumin material. Two tests were performed, the murine local lymph node assay (LLNA) and the intracutaneous reactivity test. Both of these tests are standard tests of biocompatibility that are used in the development of materials for medical devices such as surgical sutures. These tests were also performed in accordance with Good Laboratory Practice (GLP) regulations.

The LLNA test evaluated the skin sensitization potential of denatured albumin by administering an extract of denatured albumin to the skin of mice and measuring the proliferation of cells in lymph nodes draining the exposure site.

Representative portions of the denatured albumin strips were cut into pieces, placed into test tubes and prepared at a ratio of 60 cm² to 20 ml of extraction vehicle. Two different extract vehicles were used: 0.9% normal saline (NS) and dimethylsulfoxide (DMSO). Three doses of extract were prepared for each extract vehicle represented by three different denatured albumin sample surface areas per ml extract volume. The denatured albumin samples were extracted at 37° C. for 72 hours. The albumin extracts were then cooled, shaken, and decanted into sterile, dry glass vessels. Saline extracts were mixed with the detergent Pluronic L-92 to facilitate dose delivery. The extract was used within 24 hours of preparation.

Swiss mice (8 to 14 weeks old) were randomized and placed into groups of five animals each. Five mice per group were administered a 25 μl dose of albumin extract applied to the dorsum of each ear daily for three days. Five negative control mice received the same volume of vehicle administered in the same way, and five positive control mice received a known sensitizer (either 20% 2,4-dinitrobenzenesulfonic acid in NS or 0.5% dinitrochlorobenzene in DMSO). Each animal was observed daily for general health and clinical signs of toxicity according to a standard survival check paradigm. Animal weights were recorded on day 0 and day 5. Particular attention was paid to gross evidence of irritation or inflammation.

On the fifth day following dosing, each animal was injected with approximately 20 μCi of radiolabelled methylthymidine ([3H]TdR) via a tail vein injection. This isotope is rapidly incorporated into mitotically active cells (dividing lymphocytes). The isotope injection was monitored by inclusion of 0.1% Evans Blue dye for verification of delivery. The auricular lymph modes were then dissected bilaterally, isolated lymph node cells prepared, and radioactivity incorporation measured. The lymph nodes from each mouse were pooled but individual animal data were collected. Radioactivity in the lymph nodes harvested was measured and a Stimulation Index (SI) was calculated (SI=average radioactivity of albumin/average radioactivity of control). A SI greater than 3.0 indicates that the test material may be a sensitizer.

TABLE 1
Radioisotope Uptake and Stimulation Index with Saline
Extraction

| | [3H]-TdR Uptake | |
|---|---|---|
| Treatment | (DPM) | Stimulation Index |
| Negative control | 205.3 ± 94.7 | 1.0 |
| Positive control | 2706.6 ± 2613.9 | 13.18 |
| Test article extract | 300.2 ± 94.7 | 1.46 |

TABLE 2
Radioisotope Uptake and Stimulation Index with DMSO
Extraction

| | [3H]-TdR Uptake | |
|---|---|---|
| Treatment | (DPM) | Stimulation Index |
| Negative control | 497.0 ± 137.6 | 1.0 |
| Positive control | 9438.6 ± 7743.3 | 18.99 |
| Test article extract | 283.9 ± 76.7 | 0.57 |

Results of the LLNA testing showed that the denatured albumin extracts had a Stimulation Index significantly less than 3.0. Furthermore, the level of cell proliferation stimulated by the albumin extract was equivalent to the level of cell proliferation stimulated by the negative controls (Tables 1 and 2). These data demonstrated that the denatured albumin material did not produce skin sensitization and thus was biocompatible.

The intracutaneous reactivity test evaluated the skin irritation potential of denatured albumin by administering a saline and cottonseed oil extract of denatured albumin intracutaneously in rabbits and comparing the level of irritation produced locally with concurrent injections of the vehicle controls (i.e., normal saline and cottonseed oil).

More specifically, the denatured albumin material was cut into pieces, placed in test tubes, and prepared at a ratio of 60 cm² to 20 ml of extraction vehicle. Two different extract vehicles were used: 0.9% normal saline (NS) and cottonseed oil (CSO). The denatured albumin extracts and control vehicles were extracted for 72 hours at 37° C. The extracts were cooled, shaken, and decanted into a sterile, dry glass vessel. The extracts were used within 24 hours of preparation.

New Zealand White rabbits (female, >2.0 kg body weight) were randomized and housed individually. Each animal was weighed before testing and clipped on both sides of the spinal column to expose a sufficient test area for injection. Two denatured albumin extracts and two vehicle controls were injected into two rabbits.

Each rabbit received five sequential 0.2 ml intracutaneous injections of the albumin extract on the right side of the vertebral column and similar injections of the control vehicle on the left side. The second set of albumin extract and control vehicle injections were parallel and distal to the first injection sites. The animals were observed daily for abnormal clinical signs. The appearance of each injection site was noted at 24, 48 and 72 hours post injection.

The tissue reactions were rated for evidence of erythema and edema. The skin was lightly swabbed with dilute alcohol to enhance the appearance of any such reactions. The intradermal injection of CSO frequently elicits an inflammatory response. CSO scores greater than 2 are thus considered normal. Reactions were scored on a scale of 0 to 4 (0=no reaction; 1=slight reaction; 2=well-defined erythema/slight edema; 3=moderate to severe erythema/moderate edema; 4=severe erythema/severe edema) for both edema and erythema (2 scores per sites per time point). The scores for each albumin sample and control vehicle were determined and collected. Each score was divided by 12 (2 animals×3 observation periods×2 scoring categories) to determine the overall mean score for each test extract versus the corresponding control.

TABLE 3
Dermal Observations for Extraction with Normal Saline

| Rabbit | Control Scores | Test Extract Scores |
|---|---|---|
| #3654 | | |
| 24 hours | 0 | 0 |
| 48 hours | 0 | 0 |
| 72 hours | 0 | 0 |
| TOTAL | 0/6 | 0/6 |
| #3650 | | |
| 24 hours | 0 | 0 |
| 48 hours | 0 | 0 |
| 72 hours | 0 | 0 |
| TOTAL | 0/6 | 0/6 |
| COMPARATIVE RESULTS | 0 | |

TABLE 4
Dermal Observations for Extraction with Cottonseed Oil

| Rabbit | Control Scores | Test Extract Scores |
|---|---|---|
| #3654 | | |
| 24 hours | 5 | 5 |
| 48 hours | 1 | 5 |
| 72 hours | 1 | 2 |
| TOTAL | 7/6 | 12/6 |
| #3650 | | |
| 24 hours | 0 | 2 |
| 48 hours | 0 | 0 |
| 72 hours | 0 | 0 |
| TOTAL | 0/6 | 2/6 |
| COMPARATIVE RESULTS | 1.17 − 0.58 = 0.59^{A} | |

^{A}The value here is calculated by first dividing the total scores for each of the two rabbits by the number of observations (7/12 and 14/12)

The irritation reaction of the albumin extracts were compared to the vehicle controls and recorded over a 72-hour period according to the standard Irritation Scoring System. According to accepted test criteria, if the difference between the average scores for the extract of the test material and the vehicle control is less than or equal to 1.0, the test material is considered non-irritating. Results of the Intracutaneous Reactivity Test, shown in Tables 3 and 4, demonstrated that the denatured albumin extract was a non-irritant and thus would be considered biocompatible.

Considered together, the in vitro and in vivo data verify that the denatured human serum albumin product to be used as burn and wound dressing material is biocompatible. Therefore, the burn and wound dressing materials of the present invention could be considered safe for use in animals, including humans.

The denatured human serum albumin, tested above, can serve as one or more component of a burn or wound dressing material.

Single Layer Wound Dressing Embodiments

One embodiment of the burn and wound dressing material is a single sheet or lamina of the human denatured human serum albumin material, also referred to herein as “denatured albumin.” In this configuration, the lamina is rolled to a thickness of about 300 μm to about 0.5 cm in a rolling mill while interspersed between two plastic sheets.

Temporary or permanent wound dressings that are designed to enhance wound healing are needed to cover large open wounds on patients with extensive burns, lacerations and skin damage. Furthermore the ability to produce wound dressings in a variety of shapes to accommodate multiple sizes and forms of injuries is important in the manufacture of useful medical products.

The use of denatured albumin to form the wound dressing allows for the production of an unlimited number of configurations to the wound dressing. For example, in addition to lamina or sheet material, the denatured albumin material may be made into a molded dressing material. In this configuration, the 47% to 58% albumin can be dispersed inside a preset molded shape with preset differentially increased thickness for areas such as the elbow or the knee.

A common problem in the management of wounds, particularly large open wounds on patients with extensive burns and skin damage, is the management of wound exudate fluid and debris. It is important to the wound healing process that the wound dressing used allows for the removal of heavy exudate fluid and debris without dehydrating the wound bed.

The denatured albumin wound dressing allows for the removal of wound exudate fluid and debris without dehydrating the wound. A single layer manufactured denatured albumin sheet is flexible and semi-permeable to water allowing some moisture to pass through the wound dressing while preventing excessive water loss.

Alternative embodiments of the denatured albumin lamina are manufactured to include pores for the removal of exudate. The pores may be made a variety of sizes but will typically range from about 20 microns to about 200 microns. Tissue exudate and debris may be selectably removed without dehydrating the damage tissue underneath the wound dressing.

Another common problem in wound management is involved with the necessary change in dressing. For example, ordinary gauze type dressings become incorporated into the granulation tissue at the surface of the lesion so that new healthy tissue is damaged or removed when the dressing is removed. Ordinary dressings are undesirably bulky, have to be changed at frequent intervals and cause an increase in maceration with subsequent prolongation of healing time.

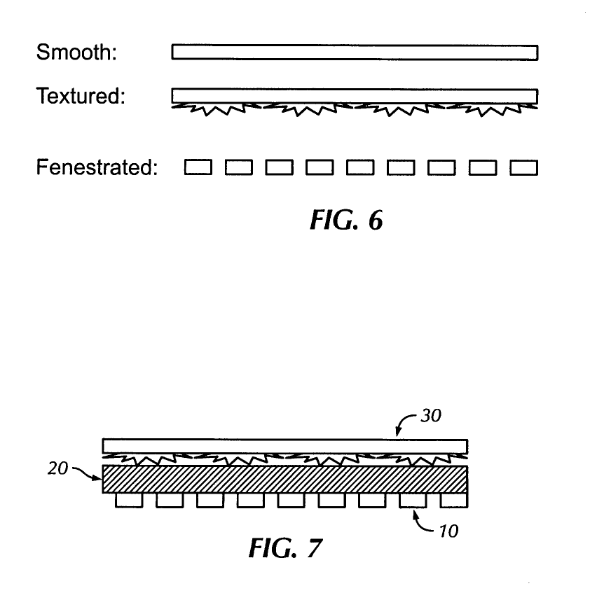

In contrast to ordinary gauze type dressings, the present flexible, non-toxic, and biodegradable denatured albumin sheet is non-irritating to the lesion and does not have to be removed. The denatured albumin lamina is applied directly to the wound. The moisture from the wound makes the surface of the denatured albumin wound dressing tacky so that after a short time period it naturally adheres to the wound. Additional configurations contemplated by the present invention involve texturing or fenestrating the interior surface (i.e., the surface that is applied to the wound) of either a denatured albumin lamina or molded dressing material as shown in FIG. 6.

Once the wound dressing is placed on the wound, it does not have to be removed. The denatured albumin wound dressing does not allow hypertrophic granulation tissue to form and helps prevent contracture of the wound. Furthermore, the denatured albumin wound dressing is relatively clear when positioned on the wound so that medical personnel can visually inspect the wound surface without removing the dressing. Thus, if an infection sets in or the wound need to be debrided, the physician can visually see the exact area that needs treatment and cut away the minimal area necessary to address the problem.

Not only can medical personnel see through the denatured albumin wound dressing, but also ultraviolet and infrared radiation penetrate the denatured albumin wound dressing. Thus, infrared radiation and/or ultraviolet therapy can be applied to the wound without removing the wound dressing.

Further embodiments of the wound dressing are impregnated with one or more agent that one of skill uses to enhance burn and wound healing, referred to herein as a “healing agent” or a “topical agent.” The denatured albumin wound dressing is impregnated with one or more healing agents by soaking the denatured albumin sheet with a solution of the agent, or by mixing the healing agent into the albumin solution used to manufacture the wound dressing.

Topical agents that are contemplated to be selectably used in the denatured albumin wound dressing include but are not limited to: (1) growth factors such as human recombinant epidermal growth factor (EGF; dose of from 1 to 1000 μg/ml; optimal dose of 500 μg/ml), vascular endothelial growth factor (VEGF), recombinant human basic fibroblast growth factor (FGF; dose of from 0.1 to 1000 ng/ml), keratocyte growth factor (KGF), platelet-derived growth factor, transforming growth factor beta, and nerve growth factor (NGF); (2) anabolic hormones such as growth hormone (GH) and human insulin (dose of from 0.001 to 10 U/ml); (3) any protease inhibitor such as nafamostat mesilate (dose of from 0.001 to 10 mg/ml); (4) any antibiotic compound at doses shown to safe and effective for human use such as a triple antibiotic (neomycin, polymyxin B, and bacitracin), neomycin, and mupirocin; and (5) the gastric pentapeptide BPC 157 (dose of from 10 to 25 μg/ml or 10 ng/kg). Also contemplated by the present invention are impregnations of the dressing material with at least one gene encoding a growth factor that can promote burn or wound healing, or alternatively impregnation of the dressing material with cells such as bone marrow mesenchymal stem cells with basic fibroblast growth factor.

Multiple Layer Wound Dressing Embodiments

The denatured albumin burn or wound dressing material can also be made of two or more layers in which one or more layers are made of denatured albumin. For example, an innermost layer of the wound dressing designed to contact the wound surface may be made a porous sheet of denatured albumin and a second layer or the wound dressing is a selectably removable sheet of denatured albumin to ensure that the wound does not dehydrate. The second layer may be selectable removed by medical personnel to allow antibiotics or other topical agents to be applied to the wound surface through the pores in the innermost layer of the dressing.

Another configuration of the wound dressing contemplated by the present invention is a sandwich type denatured albumin dressing material. In this configuration, two or three layers of denatured albumin are used in various combinations. Each layer of denatured albumin may be any one of the various embodiments of the single layer wound dressing described above.

FIG. 7 depicts an example of one such sandwich denatured albumin dressing material. The embodiment depicted in FIG. 7 has a fenestrated denatured albumin layer as the innermost layer 10 of the wound dressing, a healing agent impregnated in a second denatured albumin layer 20 or in some other material that is sandwiched between the innermost layer 10 and the outermost layer 30, and a textured denatured albumin layer as the third, outermost layer 30.

Therefore, the present invention is a biocompatible burn and wound dressing material formed from a denatured human albumin. In one embodiment, the denatured human albumin is present in the layer(s) of denatured albumin at a concentration of from 47% to 58% human serum albumin. In a preferred embodiment, the denatured human albumin has an albumin concentration of about 50% to 54%.

One of skill in the art will appreciate that in addition to the manufacturing methods described above, any industry-established method of manufacture of biocompatible burn or wound dressing materials may be used and employed with the denatured albumin material of the present invention. Further, the burn and wound dressing materials of the present invention will be used by one of skill in a variety of applications depending on the situation encountered and will include but not be limited to treatment of burns or wounds to the skin in a human patient.

It should be further appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

What is claimed is:

1. A burn and wound dressing comprising a denatured human albumin derived from denaturing a human serum albumin solution having an albumin concentration of from about 47% w/v to about 58% w/v.

2. The burn and wound dressing of claim 1 wherein the albumin concentration of the albumin solution ranges from about 50% w/v to 54% w/v.

3. The burn and wound dressing of claim 1, wherein the burn and wound dressing is sterilized with heat, gamma radiation, or ethylene oxide gas.

4. The burn and wound dressing of claim 1, wherein the burn and wound dressing has a yield strength of at least 400 kilopascals.

5. The burn and wound dressing of claim 1, wherein the burn and wound dressing has a Young's modulus of elasticity of less than 4000 kilopascals.

6. The burn and wound dressing of claim 1, wherein the burn and wound dressing is biocompatible and bioabsorbable.

7. The burn and wound dressing of claim 1 further comprising a topical agent.

8. The burn and wound dressing of claim 7, wherein the topical agent is selected from the group consisting of a growth factor, an anabolic hormone, a protease inhibitor, an antibiotic, a gastric pentapeptide BPC, and a stem cell.

9. The burn and wound dressing of claim 1, wherein the burn and wound dressing is penetrable by visible, infrared, and ultraviolet radiation.

10. A biocompatible burn and wound dressing comprising:
a first sheet of dressing material including at least 47% w/v denatured human serum albumin; and
a second sheet of dressing material including a topical agent.

11. The burn and wound dressing of claim 10, wherein the first sheet of dressing material is derived from denaturing a human serum albumin solution having an albumin concentration of from about 47% w/v to about 58% w/v.

12. The burn and wound dressing of claim 10, wherein the first sheet of dressing material has a multitude of pores traversing the sheet.

13. The burn and wound dressing of claim 10, wherein the topical agent is selected from the group consisting of a growth factor, an anabolic hormone, a protease inhibitor, an antibiotic, a gastric pentapeptide BPC, and a stem cell.

14. The burn and wound dressing of claim 10, wherein the burn and wound dressing contains more than one topical agent.

15. The burn and wound dressing of claim 10, wherein the burn and wound dressing is penetrable by visible, infrared, and ultraviolet radiation.

16. The burn and wound dressing of claim 10, wherein the burn and wound dressing is penetrable by visible, infrared, and ultraviolet radiation

17. The burn and wound dressing of claim 10, further comprising a third sheet of dressing material.

18. The burn and wound dressing of claim 17, wherein at least two sheets of the dressing material include at least 47% denatured human serum albumin.

19. A biocompatible burn and wound dressing comprising at least one sheet of dressing material containing a denatured human serum albumin at a concentration of at least 50% w/v, wherein the dressing material has a yield strength ranging from about 800 kilopascals to about 1200 kilopascals and a Young's modulus of elasticity ranging from about 2500 kilopascals to about 3500 kilopascals.

20. The burn and wound dressing of claim 19, further comprising a topical agent selected from the group consisting of a growth factor, an anabolic hormone, an antibiotic, a protease inhibitor, a gastric pentapeptide BPC, and a stem cell.

21. A biocompatible burn and wound dressing comprising:
a first sheet of porous dressing material including at least 47% w/v denatured human serum albumin; and
a second sheet of dressing material penetrable by visible light, ultraviolet radiation and infrared radiation.

22. The burn and wound dressing of claim 21 further comprising a source of mesenchymal stem cells and fibroblast growth factor.

23. The burn and wound dressing of claim 21 further comprising a source of a healing agent selected from the group consisting of an antibiotic, a growth hormone, a protease inhibitor, and an anabolic hormone.

24. A method for making a burn and wound dressing comprising the steps of:
obtaining an albumin solution having a concentration of human serum albumin ranging from about 47% w/v to about 58% w/v;
casting the albumin solution into a predetermined shape; and
denaturing the human serum albumin by heating the albumin to at least 85° C. at a pressure of at least 1 atmosphere for at least 15 seconds.

25. The method of claim 24, wherein the human serum albumin is denatured by heating the albumin solution from 15 seconds to 30 minutes between 85° C. and 120° C. at a pressure ranging from about 1 atmosphere to about 3 atmospheres.
